(19) United States
(12) Patent Application Publication
(10) Pub. No.: US 2010/0025245 A1
(43) Pub. Date: Feb. 4, 2010

(54) SELECTIVE FLUORESCENT LABELING OF S-NITROSOTHIOLS (S-FLOS): A NOVEL METHOD FOR STUDYING S-NITROSYLATION

(75) Inventors: Robert N. Cole, Baltimore, MD (US); Dan E. Berkowitz, Baltimore, MD (US); Lakshmi Santhanam, Baltimore, MD (US); Artin Andrew Shoukas, Baltimore, MD (US)

Correspondence Address: VENABLE LLP, P.O. BOX 34385, WASHINGTON, DC, 20043-9998, US

(73) Assignee: Johns Hopkins Univeristy, Balitmore, MD (US)

(21) Appl. No.: 12/442,329

(22) PCT Filed: Sep. 20, 2007
(86) PCT No.: PCT/US07/20328

§ 371 Date: Apr. 24, 2009

(30) Foreign Application Priority Data
Sep. 20, 2006 (US) 60845944

(51) Int. Cl. G01N 33/68; G01N 33/559

(52) U.S. Cl. 204/461; 436/86

(57) ABSTRACT

A method and kit for selectively labeling S-nitrosylated cysteines in proteins with a fluorescent tag. The method offers femtomolar sensitivity for the detection, quantification, in situ visualization, and a means for site-specific identification of S-nitrosylation events.

The work leading to this invention was supported by grants from the U.S. Government, NIH RO1 AG 021543 from the National Institutes of Health and the National Heart, Lung, and Blood Institute (contract N01-HV-28180). The U.S. Government has certain rights in the invention.

### BACKGROUND OF THE INVENTION

1. Field of the Invention

The invention relates to a method for selectively labeling S-nitrosylated proteins with a fluorescent tag. The method offers femtomolar sensitivity for the detection, quantification, in situ visualization, and a means for site-specific identification of nitrosylation events

2. Background Information

Protein S-nitrosylation, a reversible post-translation modification of cysteines, affects many cell signaling pathways^{1,2}. Emerging evidence suggests that dysregulation of this redox-sensitive modification is a marker of, or contributes to the pathophysiology of many disease processes including arthritis, pre-eclampsia, asthma, and stroke^{3,4}.

Rapid and global detection of biologically relevant nitrosylated proteins would help identify novel NO signaling pathways and molecular mechanisms of many redox-sensitive pathophysiologies^{5,6}. The biotin switch assay² has been used to study S-nitrosylation in a variety of proteomes⁷⁻¹⁰. It involves three steps aimed at replacing the cysteine linked nitrosothiol with a biotin tag at the S-nitrosylation sites: (1) block free thiols, (2) selectively reduce S-nitrosylated cysteines, and (3) biotinylate the newly released cysteine thiols (FIG. 1). The nitrosylated proteins are then detected by western blotting for biotin or enriched using streptavidin resins or anti-biotin antibodies for proteomic applications. Gross and coworkers developed the SNOSID assay to map sites of nitrosylation in complex mixtures^{6,11}. This is performed by trypsinizing biotinylated samples and using tandem MS to identify the peptides binding to an avidin column. The biotin switch assay has also been modified to fluorescently stain S-nitrosylated proteins in situ; streptavidin-FITC to visualize nitrosylated proteins after subjecting them to biotin switch in situ¹² or ethylmethanethiosulfonate conjugated Texas red instead of biotin-HPDP to stain nitrosylated proteins in endothelial cells¹³. However, neither of these fluorescent approaches is compatible with 2D gel electrophoresis. Thus, while the biotin-switch assay is a powerful method to identify and map protein S-nitrosylation, the method has some drawbacks: 1) it may yield false positives from endogenously biotinylated proteins, a problem that is particularly relevant in in situ staining experiments involving streptavidin-FITC¹⁴; 2) comparing relative changes in S-nitrosylation between samples is difficult and indirect; 3) it is not compatible with standard reducing one-dimension or two-dimension SDS-PAGE because the disulfide-linked biotin tag is removed by β-mercaptoethanol or DTT (FIG. 1); and 4) the avidin binding enrichment step may lead to additional false positives by co-isolation of nonnitrosylated interacting proteins.

Accordingly a new and improved method for the detection and identification of nitrosylated proteins is desirable.

### SUMMARY

We describe herein a modification of the biotin switch assay; Selective Fluorescent Labeling Of S-nitrosothiols (S-FLOS, FIG. 1). S-FLOS provides the following improvements over existing methods: 1) reduced false positives by using an exogenous synthetic fluorescent tag; 2) compatibility with 2D gel electrophoresis; 3) detection and quantification of changes in protein S-nitrosylation on a single 2D gel; 4) in situ staining; and 5) direct identification of S-nitrosylated protein with potential to map S-nitrosylation sites. Finally, the method can be extended to other column or mass spectrometry based applications for high-throughput determination of nitrosylated proteins/sites.

Accordingly, a method is provided for detecting and/or identifying S-nitrosylated protein in a sample, comprising the steps of

a) treating the sample with an alkylthiolating agent conditions such that free thiol groups in the protein become alkylthiolated;

b) removing unreacted alkylthiolating agent from the sample;

c) treating the sample with a reducing agent such that nitrosothiol bonds on the protein are reduced to form new free thiol groups;

d) treating the sample with a maleimide-derivatized fluorescent dye under conditions such that free thiol groups become labeled with said dye to form detectably labeled protein;

e) detecting/identifying detectably labeled protein, wherein said detectably labeled protein corresponds to S-nitrosylated protein contained in said sample.

The method results in a treated sample that can be further processed to quantify and map sites of S-nitrosylation within the S-nitrosylated protein, e.g. by the use of gel electrophoresis, liquid chromatography, mass spectrometry, or cytohistochemistry.

In one embodiment, the alkylthiolating agent is methyl methanethiosulfonate (MMTS). Additional suitable alkylthiolating agents will be known to those of skill in the art.

The maleimide-derivatized fluorescent dye is typically selected from the group consisting of Cy-maleimide dyes, Alexa Fluors, Texas Red, and BODIPY. Other suitable maleimide-derivatized fluorescent dyes known in the art may also be used, and can be tested for suitability without undue experimentation. In particular, Cy3-maleimide dye and/or Cy5-maleimide dye are known to produce superior results.

Any suitable reducing agent that is known to those of skill in the art can be used in the method, e.g. ascorbate.

Sodium dodecyl sulfate (SDS) and/or other suitable detergent(s) can be used along with the other reagents to ensure access of alkylthiolating agent to buried cysteines. Under the conditions used, alkylthiolating agent does not react with nitrosothiols or preexisting disulphide bonds.

Also provided is a kit for the detection/identification/quantification of S-nitrosylated protein and sites of S-nitrosylation within the S-nitrosylated protein in a sample. The kit comprises suitable reagents for carrying out the methods disclosed herein, for example, a maleimide-derivatized fluorescent dye (e.g. Cy-maleimide dyes, in particular Cy-3 and Cy-5, Alexa Fluors, Texas red, and BODIPY), and any combination of the following: an alkylthiolating reagent, a reducing agent (e.g. ascorbate), a standard sample comprising at least one protein substrate, a column cartridge for enriching Cy labeled peptides, and instructions and/or software to quantify or map S-nitrosylation sites. The kit may also comprise reagents for further processing, e.g. for 2 dimensional gels, mass spectroscopy, etc.

Methods disclosed herein have several advantages over previously used methods. First, false positives are inherently reduced. Second, fluorescent label remains present and is compatible with all protein separation techniques, including 2D gel electrophoresis, and provides means to directly identify, detect and quantify changes in S-nitrosylation on individual proteins in complex mixtures in a true multiplex format. Furthermore, the features of S-FLOS, two sample multiplexing and the ability to analyze samples by gel electrophoresis, liquid chromatography, mass spectrometry, and cytohistochemistry under reducing conditions, makes the S-FLOS method uniquely different from all other published methods. S-FLOS is a powerful cross-platform analytical and quantitative technique for S-nitrosylation and can be readily modified for analyzing other modifications of cysteines. In addition, S-FLOS has the advantage of mapping and quantify changes in nitrosylation at specific cysteines within a protein.

The methods disclosed herein can be used, or example, to screen for potential drugs which are useful in modulating protein nitrosylation, and/or to identify proteins which are affected by nitrosylation. For example, a test compound can be contacted with a biological sample and the effect of the test compound on the nitrosylation of proteins within the biological sample can be determined, e.g. by comparing nitrosylation of proteins within a biological sample to the nitrosylation of proteins within a control sample which has not been treated or contacted with the test compound. An increase or decrease in the amount of nitrosylation observed in the test sample can be used as an indication of potential usefulness as a drug for modulating protein nitrosylation. Drugs identified in this manner are expected to be useful for modulating such processes as apoptosis, neurotoxicity, neurotransmitter release, cellular proliferation, smooth muscle relaxation, and differentiation. Similarily to drug screening, these methods can be used to screen for diseases the affected by the nitrosylation or for nitrosylation state markers indicating onset or prognosis of reoxiditive related diseases.

While specific examples have been provided, the description herein is illustrative and not restrictive. Any one or more of the features of the described embodiments can be combined in any manner with one or more features of any other embodiments in the present invention. Furthermore, many variations of the invention become apparent to those skilled in the art upon review of the specification. The scope of the invention should, therefore, be determined not with reference to the description herein, but instead should be determined with reference to the appended claims along with their full scope of equivalents.

This application claims priority to U.S. provisional patent application No. 60/845,944, filed Sep. 20, 2006, which is incorporated herein by reference.

### BRIEF DESCRIPTION OF THE DRAWINGS

FIG. 1: Comparison of the biotin-switch and S-FLOS labeling schemes.

FIG. 2: S-FLOS can detect and quantify nitrosylated proteins on SDS-PAGE. A) S-FLOS analysis shows increased fluorescence of nitrosylated proteins in a mix of four known proteins nitrosylated using 50 and 100 μM GSNO. These increases were despite reduced protein recovery in GSNO treated samples (right panel, Coomassie blue stain). Cy3-(left panel) or Cy5-maleimide (middle panel) labeling shows the same result; B) Relative changes in fluorescence from four proteins in FIG. 2A were quantified using Image Quant and normalized to protein content from densitometry of Coomassie blue stained bands. Bars represent averages from 3 Cy3- and 3 Cy5-labeled samples. Error bars represent ± standard deviation of three independent experiments.

Fluorescence intensity for lysozyme was not detected, suggesting that lysozyme is not nitrosylated under these conditions; C) BSA-SNO content for each dose was quantified using an amperometric assay for total NO content. S-FLOS intensities were calculated using ImageQuant and normalized to the protein load based on densitometry silver stain images. S-FLOS can detect femtomole levels of SNO. Error bars represent ± standard deviation for three replicates from 3 Cy3- and 3 Cy5-labeled samples. Lower panel shows gel images of BSA-SNO content using S-FLOS; D) S-FLOS was used to determine endogenous nitrosylation in WT and NOS1 knockout mice (left panels). Both Cy3 and Cy5 show similar labeling with very low background in ascorbate deficient lanes (-Asc). Protein load was visualized using silver staining (right panels).

FIG. 3: S-FLOS can be used to quantify relative changes in S-nitrosylation and identify nitrosylated proteins. RAW264.7 cells were stimulated with 100 U/ml IFNγ/5 μg/ml LPS to induce NOS2-dependent NO production, thereby stimulating endogenous nitrosylation of proteins. A) Time dependent increase in NO production was confirmed using the Griess assay; B) Cells treated with IFNγ/LPS at 24 hr time point. Stimulated RAW264.7 cells and unstimulated controls were assayed with S-FLOS and labeled with Cy3 (top panel) or CyS (middle panel), respectively, then mixed and resolved on a single 2D gel. The Cy3 image shows a clear increase in nitrosylation levels compared to the control (Cy5 image). Post-staining with silver (bottom panel) shows that only about 20% of proteins are nitrosylated; C) Time-dependent increases in 21 endogenously nitrosylated proteins in RAW264.7 cells were quantified using S-FLOS. Cells treated with IFNγ/LPS were harvested at 0, 6, 24 and 48 h. As in B, each time point was subjected to S-FLOS, using Cy3 for stimulated cells and Cy5 for controls. Proteins were analyzed using 2D GE. The data are averages of three independent experiments. Error bars are omitted for clarity (*p<0.01, † p<0.05).

FIG. 4: Comparison of biotin switch and S-FLOS in raw cells. A) Proteins from lysates of RAW264.7 cells treated with exogenous NO donor (100 μM GSNO) and subjected to S-FLOS show a clear increase in fluorescence compared to the untreated samples. Swapping Cy dyes in the S-FLOS assay shows no dye dependent effect. Pretreatment of the samples with ascorbate (“Asc+” lanes 5 and 6) to eliminate SNOs results in complete loss of signal, even though these lanes were scanned at a high PMT for increased sensitivity; B) protein load was identical in all lanes as determined using coomassie blue staining; C) Biotin switch assay was performed on the same samples for comparison. After biotinylation, nitrosylated proteins were enriched using streptavidin coated agarose, resolved using SDS-PAGE, and silver stained. While the GSNO treated versus untreated lanes show similar differences as S-FLOS, pre-treatment with ascorbate (lane 3) did not eliminate signal completely.

FIG. 5: Biological replicate and dye swap of cells treated with IFNγ/LPS at 24 hr time point. S-FLOS analysis was performed as in FIG. 3B, in contrast, Cy5 was used for stimulated cells whereas Cy3 label used for unstimulated control cells. Similar background resulted with clear increases in S-FLOS fluorescence signal in proteins from the stimulated cells. Protein identifications performed on spots excised from the 2D gel in FIG. 3B.

FIG. 6: In situ staining of S-nitrosylated proteins using S-FLOS. RAW264.7 cells grown on fibronectin-coated coverslips were treated with IFNγ/LPS to induce nitrosylation, then fixed, permeabilized, and subjected to S-FLOS labeling in situ. There is a time dependent increase in SNO signal (0, 24, 48 h). The presence of the NOS2 inhibitor 1400 W abrogates the signal at 48 h (+1400 W 48 h). Pretreatment of the 48 h time point with DTT to remove all nitrosylation leads to complete loss of signal (+DTT 48 h). Similarly, omission of the ascorbate reduction step (-Asc 48 h) following MMTS blocking leads to no signal. This confirms that S-FLOS labeling is selective. Finally, staining nuclei with DAPI (blue, bottom panels) and merging these images with the nitrosylation signal (red) highlights the increase in cytoplasmic S-nitrosothiol content in IFNγ/LPS-stimulated cells at 48 h (48 h merge), whereas basal nitrosylation was low and confined to nuclei (0 h merge). The scale bars are ˜10 μm.

### DETAILED DESCRIPTION

### Definitions

By “alkylthiolating agent” is meant an agent that forms alkylthiol groups when reacted under suitable conditions with free thiol groups. Alkylthiolating agents contain straight or branched chain lower alkyl (C₁-C₆) groups that may be derivatized or functionalized, and may contain regions of unsaturation, for example MMTS. The blocking agent is preferably removed from the test sample prior to the step of the detectable tagging. MMTS, for example, can be removed by acetone precipitation (MMTS remains in the supernatant) or by subjecting the test sample to a spin column or spin filter.

Unless indicated otherwise by context, by “sample” or “test sample” is meant any sample which may be suitably tested using the methods disclosed herein. Test samples can be e.g. in the form of any biological sample, for example, crude, purified or semipurified lysates of tissues that potentially comprise nitrosylated proteins, e.g. brain, peripheral nerve, muscle, blood vessels, blood cells, liver, etc.

By “reducing agent” is meant a compound such as ascorbate that reduces nitrosothiol bonds on the protein to form new free thiol groups. Agents such as Cu²⁺ or Hg²⁺ may also be used. Care must be taken to remove these, as these metals can interfere with the labeling step.

### Methods

Animals: NOS1 knockout and wild type mice (9-11 weeks old) were used in this study and purchased from Jackson Labs. The mice were anesthetized and perfused with normal saline to remove blood. The brains were then dissected, snap frozen, and stored at −80° C. until all samples were collected (2 days). The samples were homogenized in 50 mM Tris-HCl buffer (pH 7.5) containing protease inhibitors (Roche) and 1 mM neocuproine (Sigma) and used immediately.

S-FLOS method for purified proteins: 100 μg protein were incubated with 20 mM methyl methanethiosulfonate (MMTS) at 50° C. to block the free cysteines. Excess GSNO and MMTS were removed by cold acetone precipitation and proteins were redissolved in 100 μl Reducing Buffer (50 mM TRIS-HCl pH 7.4, 4% CHAPS, 5 mM ascorbate. After a 1 h incubation, the reduced proteins were buffer exchanged in 120 μl Labeling Buffer (50 mM TRIS-HCl pH 7.0, 7 M Urea, 4% CHAPS) using protein desalting columns (Pierce). The reduction and labeling steps were performed sequentially because ascorbate sometimes interfered with the Cy-dye labeling step. 5 μl of each sample were labeled with 10 pmol of Cy3- or Cy5-maleimide, resolved using SDS-PAGE and scanned to determine fluorescence intensities (Typhoon scanner, GE Healthcare). The entire assay was performed in the dark. The gels were post stained using colloidal coomassie blue and scanned to determine protein loads using densitometry (UMAX PowerScan III).

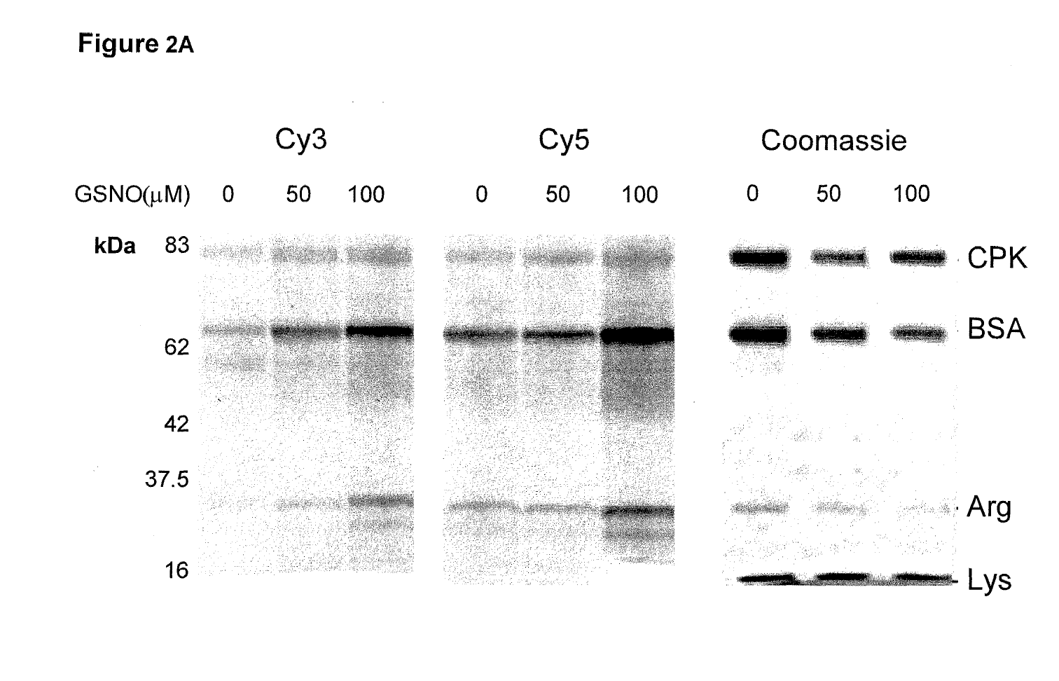

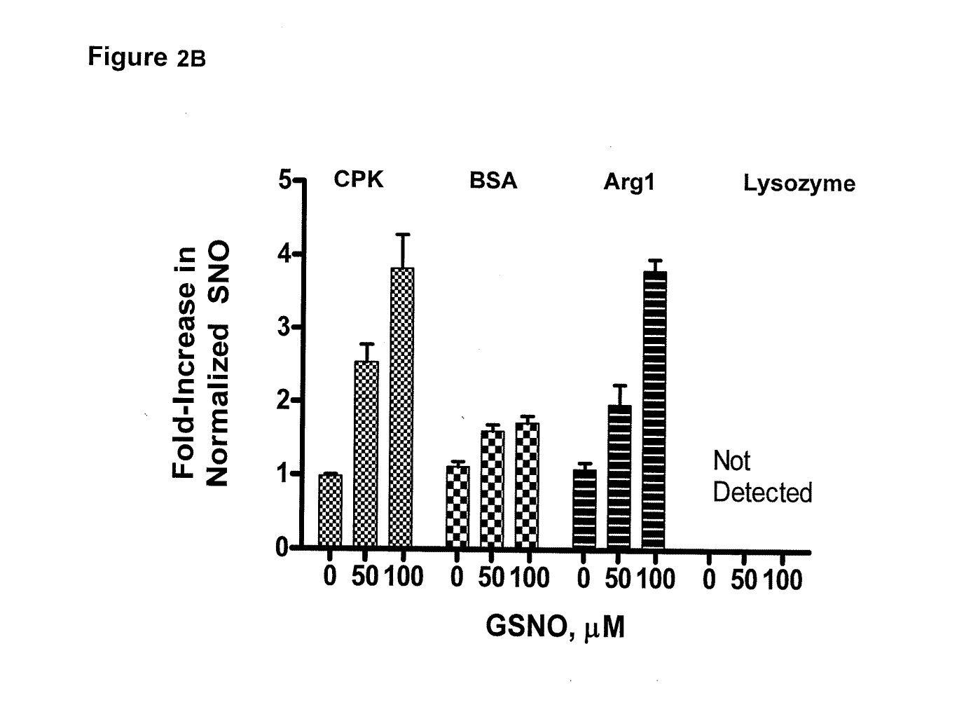

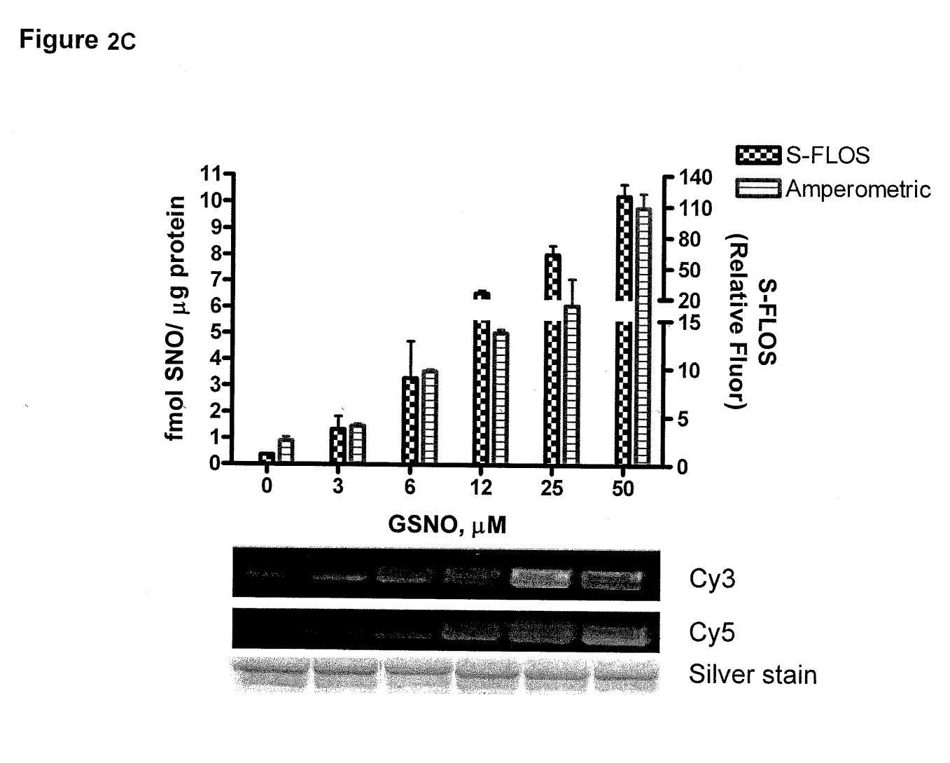

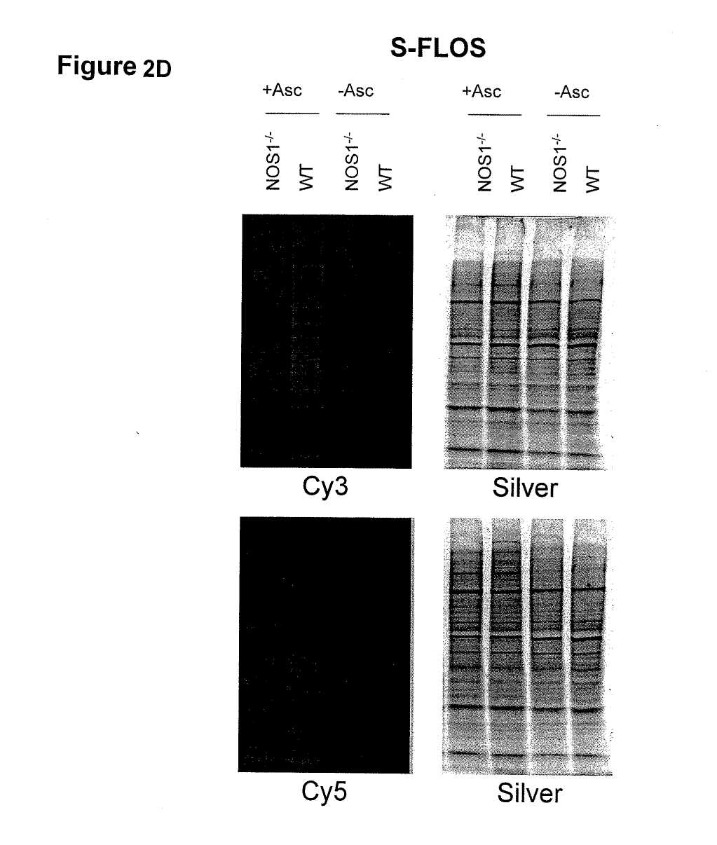

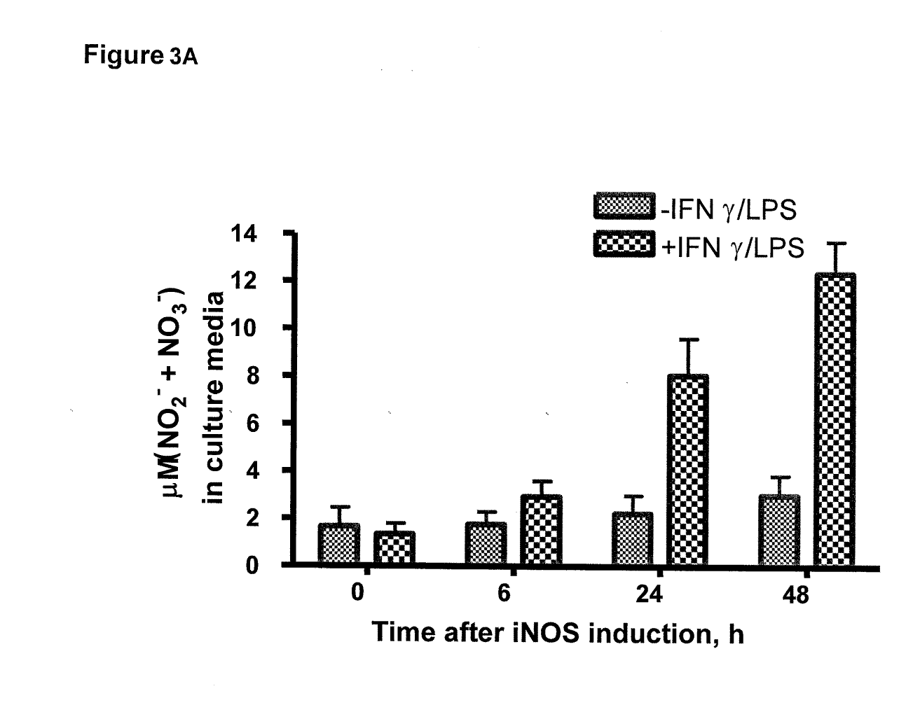

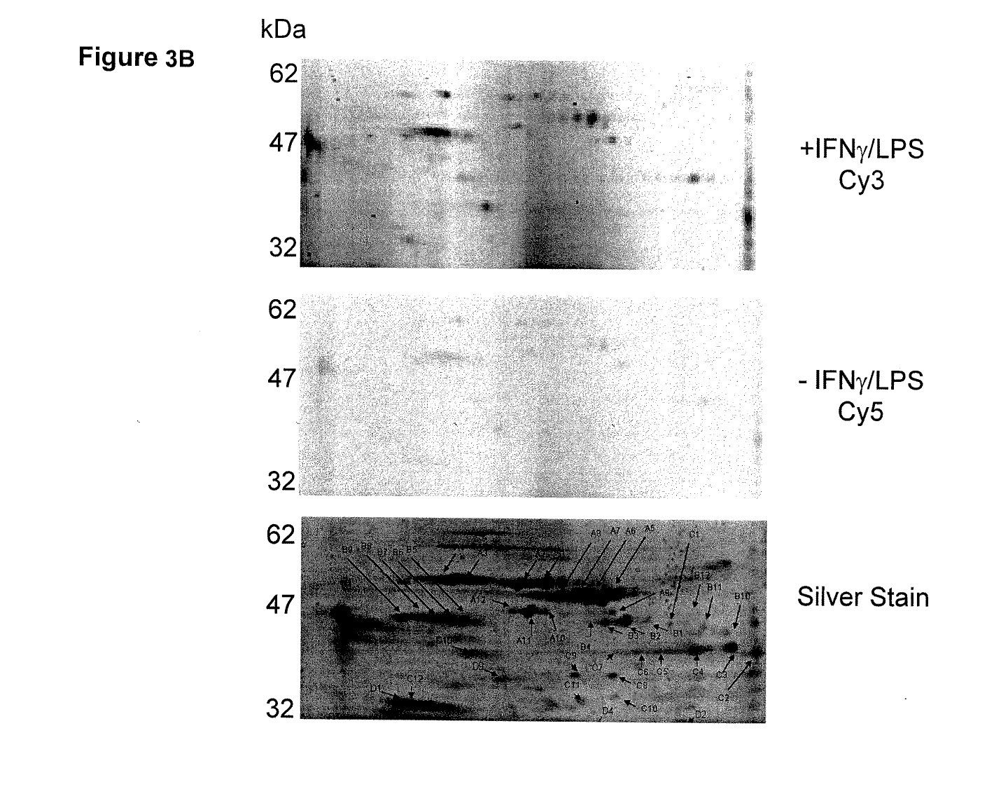

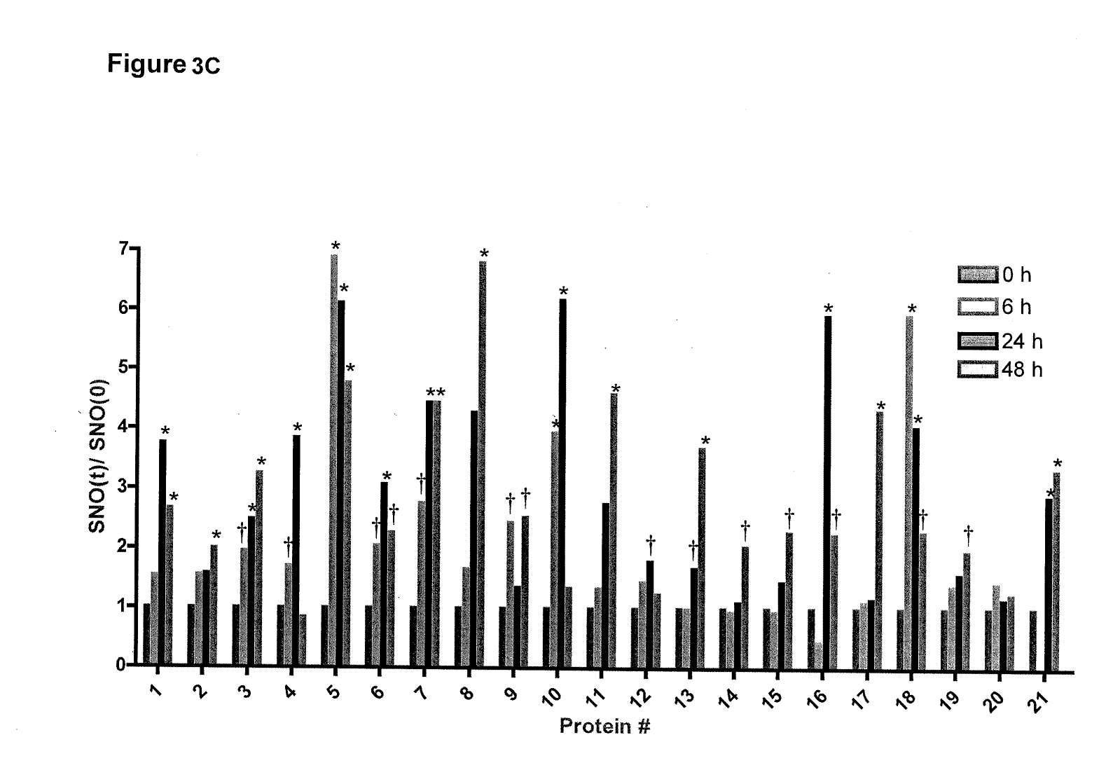

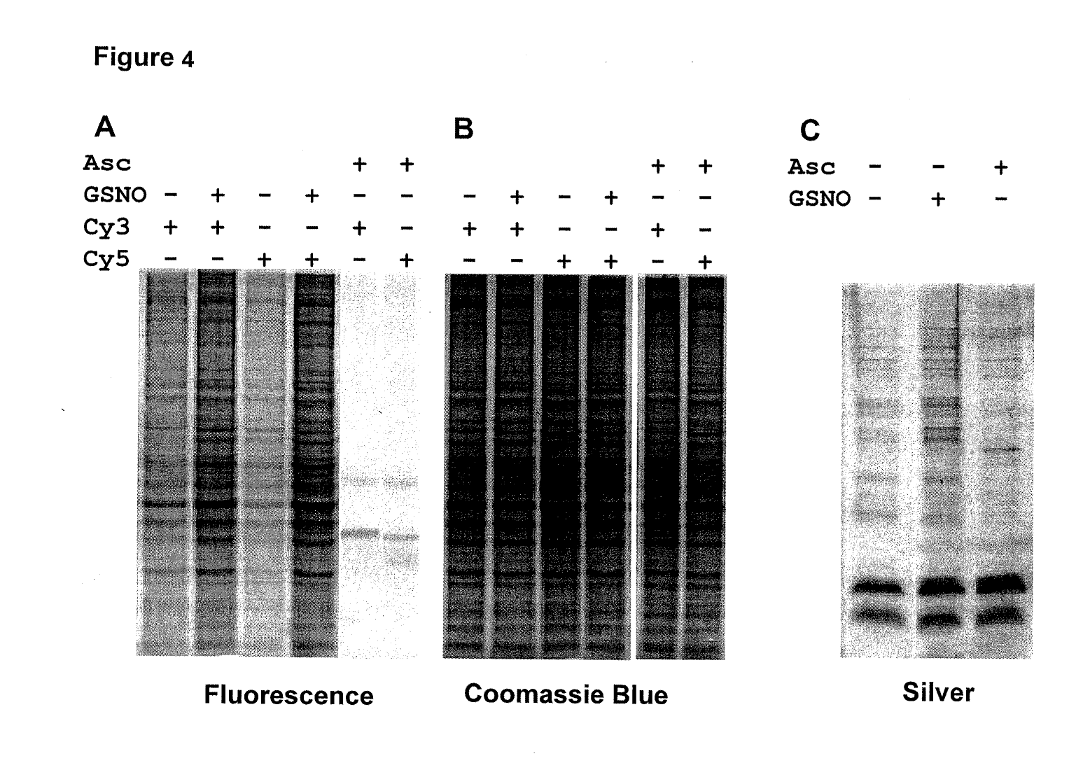

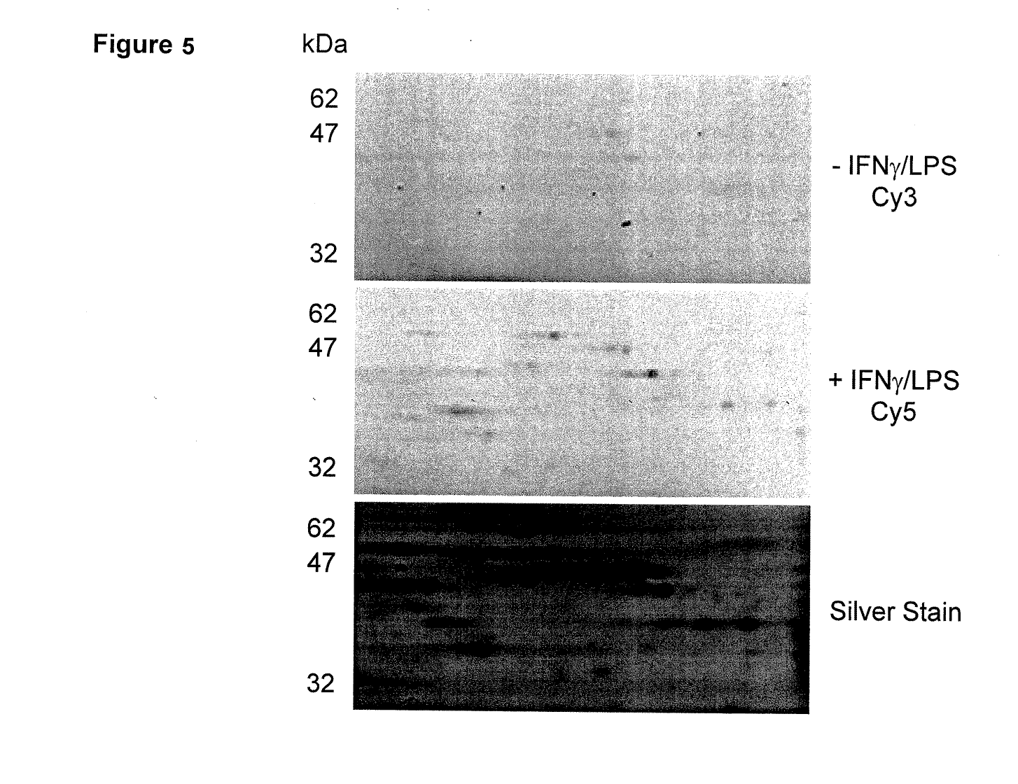

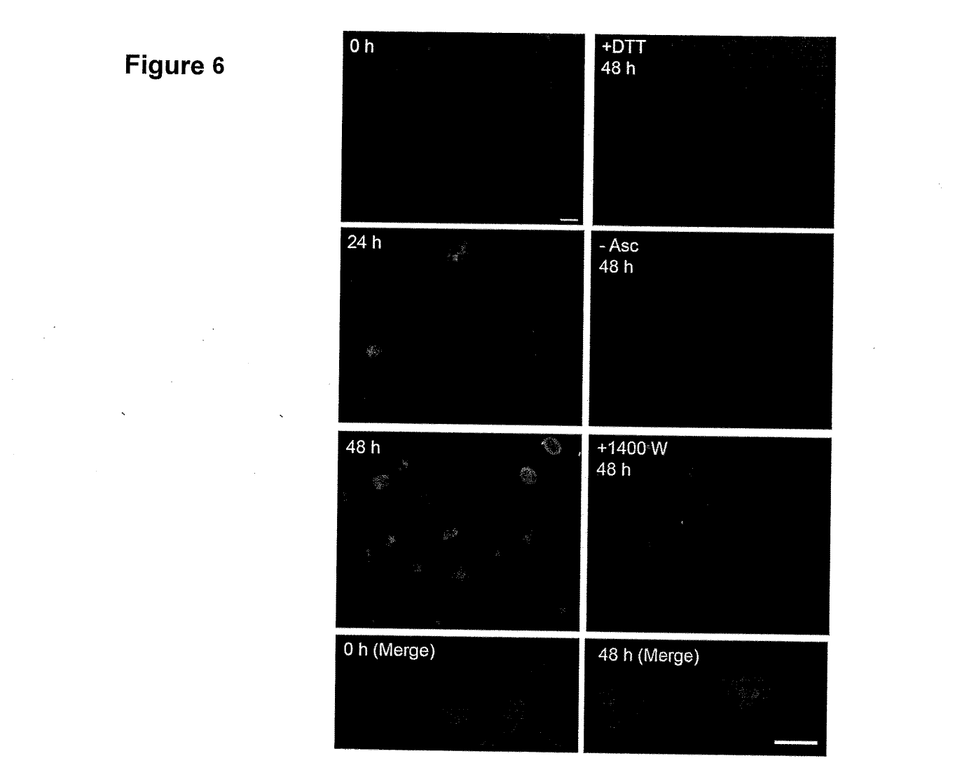

S-FLOS method for cell lysates/tissue homogenates: 100 μg total protein were blocked and reduced as described above. After buffer exchange into Labeling Buffer, protein concentration was determined either using the BioRad protein assay reagent or using 2D-gel protein Quant kit (GE Healthcare). 12.5 μg of sample was then labeled with 40 pmol of either Cy3- or Cy5-maleimide. The samples were then either resolved using SDSPAGE (FIG. 2D) or for direct comparison, the two samples were mixed and resolved using either 2D gel electrophoresis (FIG. 3B). The entire assay was performed in the dark.

2D Gel electrophoresis: 12.5 μg each of the Cy3 and Cy5 labeled samples were mixed and buffer exchanged into 120 μl of Rehydration Buffer (8 M Urea, 4% CHAPS, 0.2% DTT, 0.0002% bromophenol blue). 1.8 μl of ampholytes (pH 4-7 IPG buffer, GE Healthcare) were added, and the proteins were resolved on a 7 cm pH 4-7 IPG strip (GE Healthcare) followed by 4-12% gradient SDS-PAGE (NuPage, Invitrogen). The gels were run in the dark. Gels were imaged on a Typhoon scanner and post-stained with silver. The silver stained gel was imaged on a UMAX PowerScan III. Indicated spots were excised for identification using MS.

In situ labeling of S-nitrosothiols: IFNγ/LPS-stimulated and unstimulated RAW264.7 cells grown on coverslips were fixed in 3% paraformaldehyde and permeabilized with 0.05% Triton X-100. After blocking free thiols with 20 mM MMTS, the coverslips were washed in Wash Buffer (PBS containing 0.5% v/v Tween-20) and the SNOs reduced using 5 mM ascorbate. The cells were then rinsed in Wash Buffer and incubated with 10 μM Cy3 in Labeling Buffer at 37° C. for 30 min. All steps were performed in the dark. The samples were washed in Wash Buffer and fluorescence images were acquired using a Nikon Eclipse TE200 microscope and an internally-cooled 12-bit CCD camera (CoolSnapHQ, Photometrics, Tucson, Ariz.). Images were collected using OpenLab software (Improvision, Lexington, Mass.). Nuclei of cells were labeled with DAPI (Invitrogen) and imaged using fluorescence microscopy. Images of S-FLOS (Cy3, red) were merged with images of nuclei (blue).

Amperometric detection of S-nitrosothiols: Absolute levels of S-nitrosothiols were determined using an amperometric NO probe (WPI Inc). In brief, the ISO-NOP70L probe was polarized per vendor's protocol. GSNO was used to calibrate the probe. 200 μg BSA were treated with increasing doses of GSNO for 30 min in dark. Excess GSNO was removed by an acetone precipitation step followed by desalting (Pierce) and recovered into 120 μl. Protein concentration was determined (BioRad Protein Assay reagent). BSA-SNO levels were determined amperometrically using 100 μg of protein.

Nitrite accumulation in cell culture media: Nitrite accumulation was determined using the Nitrite/Nitrate assay kit (Calbiochem) following manufacturer's instructions.

Protein Identification: Silver stained protein bands or spots excised from gels were destained using 30 ul of 1:1 mixture of 30 mM potassium ferricyanide and 100 mM sodium thiosulfate according to Gharahdaghi et al., 1999 Electrophoresis 20, 601-5) and digested with trypsin (sequencing grade, Promega) in 20 mM ammonium bicarbonate at 37° C. overnight as previously described (Shevchenko et al., 1996 Anal Chem 68, 850-8). Extracted peptides were fractionated on a 5-40% acetonitrile gradient in 0.1% formic acid over 25 min at 300 nl/min on a 75 um×100 mm column with a 8 um emitter (New Objectives, Inc., www.newobjective.com) and packed with 5 μm, 120 Å C18 beads (YMC ODS-AQ, Waters Corp., www.waters.com). Eluting peptides were analyzed by collision-induced dissociation (CID) using nanoLC tandem mass spectrometry analysis on a QSTAR/Pulsar (Applied Biosystems/MDX Sciex, home.appliedbiosystems.com) interfaced with an Eksigent 2D nano-LC system (www.eksigent.com). Survey scans were acquired from m/z 350-1200 with up to three precursors selected for MS/MS using a dynamic exclusion of 30 s. A rolling collision energy was used to promote fragmentation. Peptide sequences were identified by screening the fragmentation data against the NCBI non-redundant database using in-house Mascot server and Mascot Daemon as an interface. Peptides with scores higher than Mascot's calculated 95% confidence probability threshold were considered reliable peptide sequences and are listed below for proteins in Table 1. Proteins with two or more reliable peptides were considered significant protein identifications. Table 1 shows a partial list the proteins identified from the 2D gel in FIG. 3.

TABLE 1
Subset of proteins identified by tandem mass spectrometry.
Spots were excised from gel shown in FIG. 3B (bottom panel).
S-nitrosylation (SNO) signal is
represented by Cy3 fluorescence in FIG. 3B.

| | | | |
|---|---|---|---|
| | | | SNO |
| Spot | Protein Identification | Reference | Signal |

Known targets

| | | | |
|---|---|---|---|
| A5/A6/A7 | Bovine serum albumin | Rafikova O, Rafikov R, and Nudler E, | Yes |
| | | 2002, PNAS 99 (9); 5913-5918 | |
| B2 | Protein disufide isomerase | Uehara T, Nakamura T, Yao D, Shi ZQ, | Yes |
| | | Gu Z, Ma Y, Masliah E, Nomura Y, | |
| | | Lipton SA., 2006; Nature 441:513-7 | |
| B5-7 | Cysteine proteinase inhibitor | Salvati, L., M. Mattu, M. Colasanti, | Yes |
| | | A. Scalone, G. Venturini, L. Gradoni, | |
| | | and P. Ascenzi.; 2001; Biochim. Biophys. | |
| | | Acta 1545:357-366 | |
| | 78-kDa Glucose-regulated | Moon KH, Hood BL, Kim BJ, Hardwick JP, | Yes |
| | protein (GRP78) | Conrads TP, Veenstra TD, Song BJ; 2006; | |
| | | Hepatology 44:1218-1230 | |
| C3/C4/C5 | Enolase1 | Gao C, Guo H, Wei J, Mi Z, Wai P Y and | Yes |
| | | Kuo PC; 2005, Nitric Oxide 12(2), 121- | |
| | | 126 | |

Novel Targets

| | | | |
|---|---|---|---|
| A2/A3 | Heat shock protein-70 | Contains consensus sequence | Yes |
| A4 | Heat shock 70 kD protein 5 | Contains consensus sequence | Yes |
| A11 | Heat shock protein-65 | Contains consensus sequence | Yes |

Negative controls

| | | | |
|---|---|---|---|
| D1/C12 | Nucleolar phospho-protein | Not described in literature/ no | No |
| | | consensus sequence | |
| D2 | Aldolase A | Not described in literature/ no | No |
| | | consensus sequence | |

| | | | | | |
|---|---|---|---|---|---|
| Observed | Mr(expt) | Mr(calc) | Delta | Score | Peptide |

Spot A7
albumin [Bos taurus]
gi|162648 Mass: 69248 Score: 525 albumin [Bos taurus]

| | | | | | |
|---|---|---|---|---|---|
| 507.8337 | 1013.6529 | 1013.6121 | 0.0409 | 75 | K.QTALVELLK.H |
| 582.3406 | 1162.6667 | 1162.6233 | 0.0434 | 64 | K.LVNELTEFAK.T |
| 653.3854 | 1304.7562 | 1304.7088 | 0.0474 | 57 | K.HLVDEPQNLIK.Q |
| 693.8291 | 1385.6437 | 1385.6133 | 0.0304 | 77 | K.YICDNQDTISSK.L |
| 740.4267 | 1478.8389 | 1478.7881 | 0.0508 | 84 | K.LGEYGFQNALIVR.Y |
| 756.4592 | 1510.9038 | 1510.8355 | 0.0683 | 64 | K.VPQVSTPTLVEVSR.S |
| 547.3394 | 1638.9964 | 1638.9304 | 0.0660 | 105 | R.KVPQVSTPTLVEVSR.S |

Spot B2
protein disulfide isomerase associated 3 [Mus musculus]
gi|6679687 Mass: 56586 Score: 398

| | | | | | |
|---|---|---|---|---|---|
| 439.2651 | 876.5157 | 876.4817 | 0.0340 | 50 | K.LNFAVASR.K |
| 498.2979 | 994.5813 | 994.5559 | 0.0254 | 55 | K.QAGPASVPLR.T |
| 499.2733 | 996.5321 | 996.5028 | 0.0293 | 58 | K.DASVVGFFR.D |
| 542.8030 | 1083.5914 | 1083.5600 | 0.0314 | 62 | K.YGVSGYPTLK.I |
| 596.3183 | 1190.6221 | 1190.5931 | 0.0290 | 71 | R.LAPEYEAAATR.L |
| 399.9305 | 1196.7695 | 1196.7128 | 0.0567 | 51 | K.LSKDPNIVIAK.M |
| 691.8818 | 1381.7491 | 1381.6725 | 0.0767 | 51 | K.SEPIPESNEGPVK.V |

Spot B6
serine (or cysteine) proteinase inhibitor, clade A (alpha-1antiproteinase,
antitrypsin), member 1
gi|27806941 Mass: 46075 Score: 249

| | | | | | |
|---|---|---|---|---|---|
| 430.7603 | 859.5060 | 859.4651 | 0.0409 | 54 | K.AALTIDEK.G |
| 457.7916 | 913.5687 | 913.5232 | 0.0455 | 59 | K.LNNELLAK.F |
| 584.8276 | 1167.6406 | 1167.6023 | 0.0383 | 59 | K.LSISETYDLK.S |
| 589.8390 | 1177.6634 | 1177.6230 | 0.0404 | 77 | K.LVDTFLEDVK.N |

78 kDa glucose-regulated protein [Mus musculus]
gi|1304157 Mass: 72412 Score: 124

| | | | | | |
|---|---|---|---|---|---|
| 491.2597 | 980.5049 | 980.4814 | 0.0235 | 59 | K.ETAEAYLGK.K |
| 699.4163 | 1396.8181 | 1396.7813 | 0.0368 | 65 | K.ELEEIVQPIISK.L |

Spot C3 (score more than 47)
enolase 1, alpha non-neuron [Mus musculus]
gi|12963491 Mass: 47095 Score: 154

| | | | | | |
|---|---|---|---|---|---|
| 450.2993 | 898.5841 | 898.5487 | 0.0354 | 47 | K.TIAPALVSK.K |
| 452.7520 | 903.4895 | 903.4549 | 0.0347 | 49 | R.IEEELGSK.A |
| 472.7880 | 943.5615 | 943.4974 | 0.0641 | 57 | K.VNVVEQEK.I |

Spot A2
heat shock 70 protein [Mus musculus]
gi|1661134 Mass: 70793 Score: 834

| | | | | | |
|---|---|---|---|---|---|
| 541.3075 | 1080.6005 | 1080.5603 | 0.0402 | 59 | K.LLQDFFNGK.E |
| 599.3603 | 1196.7061 | 1196.6553 | 0.0508 | 51 | K.FELTGIPPAPR.G |
| 600.3646 | 1198.7146 | 1198.6669 | 0.0476 | 56 | K.DAGTIAGLNVLR.I |
| 614.8437 | 1227.6728 | 1227.6207 | 0.0521 | 67 | K.VEIIANDQGNR.T |
| 627.3391 | 1252.6636 | 1252.6087 | 0.0549 | 72 | R.FEELNADLFR.G |
| 635.8178 | 1269.6211 | 1269.5547 | 0.0664 | 52 | R.FDDAVVQSDMK.H + |
| | | | | | Oxidation (M) |
| 652.3312 | 1302.6479 | 1302.5914 | 0.0565 | 64 | K.NSLESYAFNMK.A |
| 744.3956 | 1486.7766 | 1486.6940 | 0.0827 | 50 | R.TTPSYVAFTDTER.L |
| 833.4136 | 1664.8126 | 1664.7828 | 0.0299 | 60 | K.NQVAMNPTNTVFDAK.R + |
| | | | | | Oxidation (M) |
| 596.6901 | 1787.0484 | 1786.9828 | 0.0656 | 57 | R.IINEPTAAAIAYGLDKK.V |

Spot A4
Heat shock 70 kD protein 5 (glucose-regulated protein) [Mus musculus]
gi|29748016 Mass: 72377 Score: 618

| | | | | | |
|---|---|---|---|---|---|
| 491.2558 | 980.4971 | 980.4814 | 0.0157 | 59 | K.ETAEAYLGK.K |
| 609.3525 | 1216.6905 | 1216.6234 | 0.0672 | 88 | K.DAGTIAGLNVMR.I |
| 614.8326 | 1227.6507 | 1227.6207 | 0.0300 | 70 | R.VEIIANDQGNR.I |
| 658.8472 | 1315.6798 | 1315.6295 | 0.0502 | 53 | R.NELESYAYSLK.N |
| 699.4357 | 1396.8569 | 1396.7813 | 0.0756 | 68 | K.ELEEIVQPIISK.L |
| 715.8677 | 1429.7209 | 1429.6837 | 0.0371 | 63 | R.TWNDPSVQQDIK. F |
| 730.8991 | 1459.7837 | 1459.7518 | 0.0320 | 80 | K.SDIDEIVLVGGSTR.I |
| 559.9749 | 1676.9027 | 1676.8005 | 0.1022 | 70 | K.NQLTSNPENTVFDAK.R |
| 630.0210 | 1887.0413 | 1886.9638 | 0.0774 | 68 | K.VTHAVVTVPAYFNDAQR.Q |

Spot A11
heat shock protein 65 [Mus musculus]
gi|51455 Mass: 60903 Score: 387

| | | | | | |
|---|---|---|---|---|---|
| 393.2741 | 784.5336 | 784.5058 | 0.0278 | 57 | K.IGIEIIK.R |
| 451.2927 | 900.5709 | 900.5280 | 0.0429 | 72 | K.LSDGVAVLK.V |
| 456.8100 | 911.6055 | 911.5803 | 0.0252 | 68 | K.VGLQVVAVK.A |
| 608.3605 | 1214.7064 | 1214.6506 | 0.0558 | 67 | K.NAGVEGSLIVEK.I |
| 672.8831 | 1343.7517 | 1343.7085 | 0.0432 | 71 | R.TVIIEQSWGSPK.V |
| 549.6565 | 1645.9476 | 1645.8960 | 0.0516 | 52 | K.VGEVIVTKDDAMLLK.G + |
| | | | | | Oxidation (M) |

Spot D1 (score more than 40, spectra manually validated)
Nucleophosmin 1 [Mus musculus]
gi|55153941 Mass: 32568 Score: 147

| | | | | | |
|---|---|---|---|---|---|
| 373.2605 | 744.5064 | 744.4745 | 0.0318 | 40 | K.VTLATLK.M |
| 466.2696 | 930.5247 | 930.4658 | 0.0589 | 61 | K.GPSSVEDIK.A |

Spot D2
aldolase A [Mus musculus]
gi|7548322 Mass: 39526 Score: 307

| | | | | | |
|---|---|---|---|---|---|
| 401.2578 | 800.5010 | 800.4756 | 0.0254 | 55 | R.ALQASALK.A |
| 666.8726 | 1331.7307 | 1331.6932 | 0.0375 | 94 | K.GILAADESTGSIAK.R |

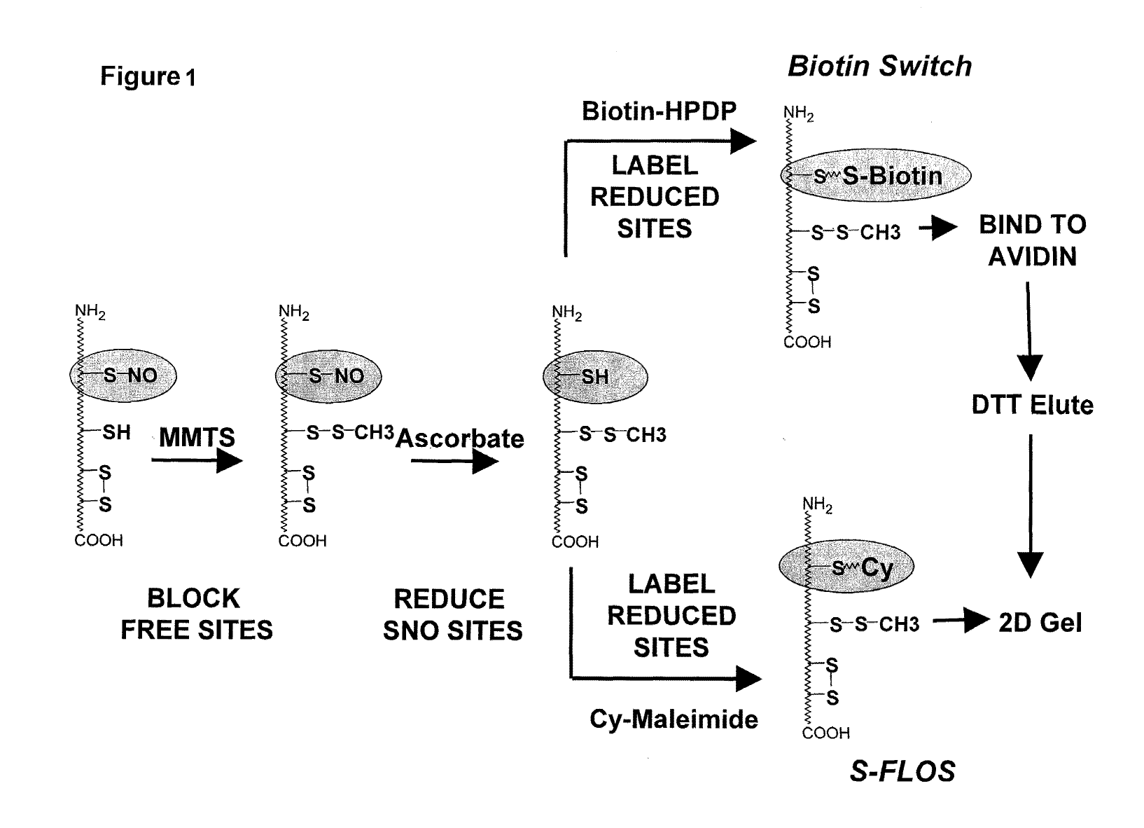

S-FLOS may use identical blocking and reduction steps as the biotin switch assay (FIG. 1). However, instead of HPDP-Biotin, S-FLOS uses maleimide conjugated Cy dyes (GE Healthcare) under conditions optimized for selectively labeling only formerly nitrosylated cysteines that are (ascorbate) reduced cysteine thiols, leaving disulfides intact. These Cy dyes have been successfully used to detect free thiols in protein samples^{13,16}, but have not been used to detect or quantify S-nitrosylated proteins. Two commercially available Cy-maleimide dyes (Cy3 and Cy5) allow labeling of two biological samples of interest with different fluorescent tags. Relative differences between the samples can then be quantified using fluorescent imaging technology. Other maleimide linked dyes such as Alexa Fluors, Texas red, and BODIPY can also be used for SDS-PAGE and in situ staining applications; however, unlike Cy dyes, these dyes are not charge balanced and, thus, are unsuitable for 2D gels.

For proof of principle, we studied the exogenous nitrosylation of a mixture of four proteins by the NO donor, S-nitrosoglutathione (GSNO) (FIG. 2A). 100 μg of a mixture of Creatine Kinase (CPK), Bovine Serum Albumin (BSA), Arginase I, and Lysozyme was incubated with increasing doses of GSNO (0, 50, and 100 μM). CPK¹⁷ and BSA¹⁸ are known targets of nitrosylation; and Arg 1 has been recently identified as a target for S-nitrosylation in this laboratory²⁰. Lysozyme served as a negative control for the S-FLOS assay. The GSNO treatment was followed by labeling nitrosylated proteins using the SFLOS assay (see detailed methods; FIG. 2A). The fluorescence intensities were calculated using Image Quant software (GE Healthcare), and normalized to protein levels (FIG. 2B). While each sample began with 100 μg of protein, reproducible losses in protein recovery occurred during the acetone precipitation step in the presence of GSNO. As seen in FIG. 2A, fluorescence intensities clearly increased in samples treated with GSNO (left and middle panels), despite the loss of protein experienced in the presence of GSNO (FIG. 2A, right panel). In addition, relative increases in fluorescence intensities were the same for either dye as demonstrated by small error bars in FIG. 2B. Therefore, SFLOS can detect and quantify relative changes in exogenously nitrosylated proteins.

We next used this method to quantify exogenously nitrosylated BSA and determined the sensitivity of the method. An amperometric probe (WPI Inc), calibrated with GSNO, was used to quantify absolute levels of BSA S-nitrosylation and detected basal nitrosylation in BSA. The S-FLOS method clearly detected changes in BSA nitrosylation at very low GSNO concentrations (<3 μM), and even detected basal nitrosylation of BSA (FIG. 2C, lower panel). In the S-FLOS experiment, each lane contained 5 μg BSA. Correlating this to the corresponding S-nitrosothiol content calculated from the amperometric assay (fmol/μg), it is clear that S-FLOS can detect femtomole levels of SNO.

We next compared S-FLOS with the biotin switch assay using IFNγ/LPS to induce NOS2 in RAW264.7 cells (FIG. 4). Pretreating proteins with ascorbate before S-FLOS or biotin switch led to a nearly complete loss of signal in S-FLOS. This was in contrast to a high background with the biotin switch (FIG. 4, compare lanes 5 and 6 in panel A with lane 3 in panel C). We detected basal levels of nitrosylation in unstimulated cells, which is consistent with literature^{7,19}.

We also compared the S-FLOS method with existing fluorescent staining methods^{12,13}. To compare its efficacy to published staining methods^{g}, we used S-FLOS to visualize S-nitrosylated proteins in fixed and permeabilized stimulated vs unstimulated RAW264.7 cells (FIG. 6). The increasing levels of S-nitrosylation observed in stimulated cells with time were clearly identified using S-FLOS (FIG. 6). Furthermore, the S-nitrosylation signal was attenuated in cells that were treated with IFNγ/LPS in the presence of the NOS2 inhibitor 1400 W. In cells that were pretreated with DTT prior to MMTS blocking to eliminate nitrosylation, virtually no signal was observed. Omission of the ascorbate reduction step after blocking free thiols similarly resulted in low signal. This confirmed that the increasing levels of fluorescent signal obtained at 24 h and 48 h after treatment with IFNγ/LPS was indeed a selective staining of S-nitrosylated proteins. There was an increase in the cytoplasmic S-nitrosothiol content of stimulated cells with time (FIG. 6, 0 h merge vs. 48 h merge). This coincided with the cytoplasmic presence of NOS2. This methodology provides consistent data compared to those from histochemical staining experiments in literature^{12,13}.

We next used S-FLOS to determine basal nitrosylation in the brain homogenates of wild-type compared to NOS1^{-/-} mice. Samples were subjected to S-FLOS and resolved using SDS-PAGE (see supplement for detailed methods). Differences in signal intensity between the wild-type and NOS1 knockout mice were clearly distinguished (FIG. 2D). Furthermore, the labeling was specific (very low levels of labeling in ascorbate deficient lanes), and was unaffected by choice of dye. NOS1^{-/-} samples are not devoid of signal because of the presence of other NOS isoforms, particularly NOS3.

We then determined the capability of S-FLOS to quantify endogenous protein S-nitrosylation (FIG. 3). RAW264.7 cells were stimulated with 100 U/ml IFNγ and 5 μg/ml LPS to induce NOS2-dependent NO production, and thereby, nitrosylation⁷. Proteins from three biological replicates were extracted at four time points over 48 h. Untreated RAW264.7 cells served as controls and were harvested at the same time intervals as the treated samples. We confirmed the production of NO by quantifying the accumulation of NO 2 and NO3 in culture media using the Griess assay (FIG. 3A). The lysates were subjected to S-FLOS. At each time point, stimulated samples were labeled with Cy3- and the unstimulated controls with Cy5-maleimide, mixed and resolved on a single 2D gel (for detailed methods, see supplement). Spots of interest were excised and identified by tandem mass spectroscopy (see supplement for detailed methods). Using this approach, we were able to detect an increase in protein nitrosylation in RAW264.7 cells treated with IFNγ/LPS when compared to untreated samples (FIG. 3B, top and middle panels, respectively). Comparison of the fluorescence (FIG. 3B, top panel) and the silver stain images (FIG. 3B, bottom panel) underscores the specificity, selectivity, and sensitivity of the S-FLOS method. Various high abundance proteins such as spots B2, C12, and D1 have no fluorescence signal whereas several low-abundance proteins (e.g., spot A9) have the fluorescent tag. As summarized in FIG. 3C, 21 spots had different time dependent increases in fluorescent intensity when stimulated with IFNγ/LPS, changes in protein nitrosylation peaked at different time points (e.g. proteins 5, 10 and 11) or there was no change (e.g. protein 20). A subset of proteins associated with increased Cy fluorescence (and hence, nitrosylation) are presented in Table 1. Five are known targets for nitrosylation. Two proteins are novel targets for S-nitrosylsation and also contain the loose consensus sequence for S-nitrosylation ((K/R/H/D/E)C(D/E))²⁰. The two proteins that did not have Cy fluorescence also do not have this consensus sequence and are not reported to be S-nitrosylated in the literature.

In conclusion, S-FLOS is a selective and sensitive assay to detect endogenous S-nitrosylation. It provides direct quantification in 2D electrophoresis applications which is not possible with any other S-nitrosylation assay described to date, while bypassing streptavidin affinity purification steps. Because the Cy-maleimide dyes label all nitrosylated protein there will be no mass shift between Cy dye and silver stain images. While S-FLOS can identify changes in S-nitrosylation, it currently does not distinguish between changes in protein expression versus changes in the number(s) of modified cysteines. In order to achieve this, a second method, such as a traditional DIGE gel²¹, must be performed to determine relative changes in protein expression. Finally, S-FLOS can be used for in situ detection of S-nitrosothiols in intact cell and tissue samples, which is in good agreement with other fluorescent methods demonstrated to date^{12,13}. This method has great potential for directly mapping S-nitrosylation sites and opens up other chromatography based applications. However, merely being able to detect quantitative differences between samples on a single 2D gel will provide extremely useful information on quantitative, spatial, and temporal changes in S-nitrosylation.

### References

References cited herein are listed below for convenience and are hereby incorporated by reference.
1 Hess, D. T., Matsumoto, A., Kim, S. O., Marshall, H. E. & Stamler, J. S. Protein S-nitrosylation: purview and parameters. Nat Rev Mol Cell Biol 6, 150-66 (2005).
2 Jaffrey, S. R., Erdjument-Bromage, H., Ferris, C. D., Tempst, P. & Snyder, S. H. Protein S-nitrosylation: a physiological signal for neuronal nitric oxide. Nat Cell Biol 3, 193-7 (2001).
3 Foster, M. W., McMahon, T. J. & Stamler, J. S. S-nitrosylation in health and disease. Trends Mol Med 9, 160-8 (2003).
4 Liu, L. et al. Essential roles of S-nitrosothiols in vascular homeostasis and endotoxic shock. Cell 116, 617-28 (2004).
5 Derakhshan, B., Wille, P. C. & Gross, S. S. Unbiased identification of cysteine Snitrosylation sites on proteins. Nat Protoc 2, 1685-91 (2007).
6 Hao, G., Derakhshan, B., Shi, L., Campagne, F. & Gross, S. S. SNOSID, a proteomic method for identification of cysteine S-nitrosylation sites in complex protein mixtures. Proc Natl Acad Sci U S A 103, 1012-7 (2006).
7 Gao, C. et al. Identification of S-nitrosylated proteins in endotoxin-stimulated RAW264.7 murine macrophages. Nitric Oxide 12, 121-6 (2005).
8 Kuncewicz, T., Sheta, E. A., Goldknopf, I. L. & Kone, B. C. Proteomic analysis of S-nitrosylated proteins in mesangial cells. Mol Cell Proteomics 2, 156-63 (2003).
9 Lindermayr, C., Saalbach, G. & Dumer, J. Proteomic identification of S-nitrosylated proteins in Arabidopsis. Plant Physiol 137, 921-30 (2005).
10 Martinez-Ruiz, A. & Lamas, S. Detection and proteomic identification of Snitrosylated proteins in endothelial cells. Arch Biochem Biophys 423, 192-9 (2004).
11 Lu, X. M., Lu, M., Tompkins, R. G. & Fischman, A. J. Site-specific detection of Snitrosylated PKB alpha/Akt1 from rat soleus muscle using CapLC-Q-TOF(micro) mass spectrometry. J Mass Spectrom 40, 1140-8 (2005).
12 Ckless, K. et al. In situ detection and visualization of S-nitrosylated proteins following chemical derivatization: identification of Ran GTPase as a target for Snitrosylation. Nitric Oxide 11, 216-27 (2004).
13 Yang, Y. & Loscalzo, J. S-nitrosoprotein formation and localization in endothelial cells. Proc Natl Acad Sci U S A 102, 117-22 (2005).
14 Varma, V. A., Cerjan, C. M., Abbott, K. L. & Hunter, S. B. Non-isotopic in situ hybridization method for mitochondria in oncocytes. J Histochem Cytochem 42, 273-6 (1994).
15 Hurd, T. R., Prime, T. A., Harbour, M. E., Lilley, K. S. & Murphy, M. P. Detection of Reactive Oxygen Species-sensitive Thiol Proteins by Redox Difference Gel Electrophoresis: IMPLICATIONS FOR MITOCHONDRIAL REDOX SIGNALING. J Biol Chem 282, 22040-51 (2007).
16 Maeda, K., Finnie, C. & Svensson, B. Cy5 maleimide labelling for sensitive detection of free thiols in native protein extracts: identification of seed proteins targeted by barley thioredoxin h isoforms. Biochem J 378, 497-507 (2004).
17 Wolosker, H., Panizzutti, R. & Engelender, S. Inhibition of creatine kinase by S-nitrosoglutathione. FEBS Lett 392, 274-6 (1996).
18 Stamler, J. S. et al. S-nitrosylation of proteins with nitric oxide: synthesis and characterization of biologically active compounds. Proc Natl Acad Sci U S A 89, 444-8 (1992).
19 Zhang, Y. & Hogg, N. Formation and stability of S-nitrosothiols in RAW 264.7 cells. Am J Physiol Lung Cell Mol Physiol 287, L467-74 (2004).
20 Stamler, J. S., Toone, E. J., Lipton, S. A. & Sucher, N. J. (S)NO signals: translocation, regulation, and a consensus motif. Neuron 18, 691-6 (1997).
21 Alban, A. et al. A novel experimental design for comparative two-dimensional gel analysis: two-dimensional difference gel electrophoresis incorporating a pooled internal standard. Proteomics 3, 36-44 (2003).
22 Santhanam, L et al. iNOS-dependent S-nitrosylation and Activation of Arginase1 Contributes to Age Related Endothelial Dysfunction. Circ Res (Accepted) (2007)

1. A method of detecting and/or identifying S-nitrosylated protein in a sample, comprising the steps of
a) treating the sample with an alkylthiolating agent conditions such that free thiol groups in the protein become alkylthiolated;
b) removing unreacted alkylthiolating agent from the sample;
c) treating the sample with a reducing agent such that nitrosothiol bonds on the protein are reduced to form new free thiol groups;
d) treating the sample with a maleimide-derivatized fluorescent dye under conditions such that free thiol groups become labeled with said dye to form detectably labeled protein;
e) detecting/identifying detectably labeled protein, wherein said detectably labeled protein corresponds to S-nitrosylated protein contained in said sample.

2. The method of claim 1 that further comprises quantifying and/or mapping sites of S-nitrosylation within the S-nitrosylated protein.

3. The method of claim 1 wherein the sample is further subjected to gel electrophoresis, liquid chromatography, mass spectrometry, or cytohistochemistry.

4. The method of claim 1 wherein the alkylthiolating agent is methyl methanethiosulfonate (MMTS).

5. The method of claim 1 wherein the maleimide-derivatized fluorescent dye is selected from the group consisting of Cy-maleimide dyes, Alexa Fluors, Texas Red, and BODIPY.

6. The method of claim 1 wherein the maleimide-derivatized fluorescent dye is Cy3-maleimide dye and/or Cy5-maleimide dye.

7. The method of claim 1 wherein the reducing agent is ascorbate.

8. A kit for the detection/identification/quantification of S-nitrosylated protein and sites of S-nitrosylation within the S-nitrosylated protein in a sample comprising a alkylthiolating reagent and a maleimide-derivatized fluorescent dye.

9. The kit of claim 8 that additionally comprises a reducing agent.

10. The kit of claim 9 wherein the reducing agent is ascorbate.

11. The kit of claim 8 that comprises a dye selected from the group consisting of Cy-maleimide dyes, Alexa Fluors, Texas red, and BODIPY.

12. The kit of claim 8 that comprises a standard sample comprising at least one protein substrate.

13. The kit of claim 8 that comprises a column cartridge for enriching Cy labeled peptides.

14. The kit of claim 8 that comprises instructions and/or software to quantify or map S-nitrosylation sites.

15. A method of detecting and/or identifying S-nitrosylated protein and the sites of S-nitrosylation within the S-nitrosylated protein in a sample, comprising the steps of
a) addition of an alkylthiolating agent to the sample under conditions such that free thiol groups in the protein become alkylthiolated;
b) removal of unreacted alkylthiolating agent from the sample;
c) addition of ascorbate to the sample under conditions such that S-nitrosylated thiol groups are selectively reduced;
d) addition of maleimide-derivatized fluorescent dye under conditions such that free thiol groups become labeled with said dye to form detectably labeled protein; wherein said detectably labeled protein corresponds to S-nitrosylated protein contained in said sample.

16. The method of claim 15, additionally comprising the step of subjecting the sample to gel electrophoresis, liquid chromatography, mass spectrometry or cytohistochemistry to identify S-nitrosylated protein(s) and the sites of S-nitrosylation within the S-nitrosylated protein.

17. The method of claim 15 wherein the alkylthiolating agent is methyl methanethiosulfonate (MMTS).

18. The method of claim 15 wherein the maleimide-derivatized fluorescent dye is selected from the group consisting of Cy-maleimide dyes, Alexa Fluors, Texas Red, and BODIPY.

19. The method of claim 15 wherein the maleimide-derivatized fluorescent dye is Cy3-maleimide dye and/or Cy5-maleimide dye.
